(19) United States
(12) Patent Application Publication
(10) Pub. No.: US 2016/0325264 A1
(43) Pub. Date: Nov. 10, 2016

(54) MULTI-CHANNEL PYROLYSIS TUBES, MATERIAL DEPOSITION EQUIPMENT INCLUDING THE SAME AND ASSOCIATED METHODS

(71) Applicant: HZO, Inc., Draper, UT (US)

(72) Inventors: James Dempster, Reno, NV (US); Jason Maynard, Salt Lake City, UT (US)

(21) Appl. No.: 15/217,678

(22) Filed: Jul. 22, 2016

Related U.S. Application Data
(62) Division of application No. 14/457,690, filed on Aug. 12, 2014, now abandoned.
(60) Provisional application No. 61/932,774, filed on Jan. 28, 2014.

(51) Int. Cl. B01J 19/24; C23C 16/455; C07C 4/24

(52) U.S. Cl. CPC B01J 19/2415; C07C 4/24; C23C 16/455; B01J 2219/24; B01J 2219/00087

(57) ABSTRACT

A pyrolysis tube for use with a material deposition system includes a plurality of channels. The channels may be defined by internal elements of the pyrolysis tube, or by internal elements that form an insert for a conventionally configured pyrolysis tube. One or more of the channels may extend straight through the pyrolysis tube, providing a direct line of sight through the pyrolysis tube. Material deposition systems that include such an insert or pyrolysis tube are also disclosed, as are methods for efficiently pyrolyzing precursor materials at temperatures that are reduced relative to conventional pyrolysis temperatures and/or at rates that are increased relative to conventional pyrolysis rates.

### CROSS-REFERENCE TO RELATED APPLICATION

This application is a division of U.S. patent application Ser. No. 14/457,690 filed on Jul. 30, 2015, titled MULTI-CHANNEL PYROLYSIS TUBES, MATERIAL DEPOSITION EQUIPMENT INCLUDING THE SAME AND ASSOCIATED METHODS (“the '690 application), which application claims the benefit of priority under 35 U.S.C. §119(e) to the Jan. 28, 2014 filing date of U.S. Provisional Patent Application No. 61/932,774, titled PYROLYSIS TUBE INCLUDING ONE OR MORE BAFFLES (“the '774 Provisional application”) is hereby made pursuant to. The entire disclosures of the '774 Provisional application and the '690 application are hereby incorporated herein.

### TECHNICAL FIELD

This disclosure relates generally to pyrolysis tubes for material processing equipment (e.g., material deposition systems, etc.) and, more specifically, to pyrolysis tubes that are configured to improve the efficiency with which molecules of a precursor material are broken down, or “cracked,” into smaller reactive species. In addition, this disclosure relates to inserts for pyrolysis tubes, pyrolysis methods and equipment for depositing or otherwise processing materials, such as parylene.

### RELATED ART

Pyrolysis is a process by which an organic material is subjected, in an environment devoid of oxygen, to a temperature that is hot enough to decompose the organic material. More specifically, when an organic material is pyrolyzed, it undergoes an irreversible physical change. Among a wide variety of other uses, pyrolysis is used to crack unsubstituted and substituted [2.2] paracyclophanes, which are also commonly referred to as “Parylene dimers”—the precursors to various types of Parylene, or polyp-xylylene)—into reactive monomers.

Parylene dimers are typically pyrolyzed in a vacuum at temperatures that are sufficient to “crack” or break apart, molecules that are introduced into the pyrolysis tube. A pyrolysis temperature of about 680° C. is typical when depositing a parylene, or a polyp-xylylene). Pyrolysis of pure parylene dimers is typically considered to be a highly efficient process; however, since some contaminants are typically present in the parylene precursor, and possibly because pyrolysis tubes are rarely totally devoid of oxygen, the process of cracking parylene dimers can be inefficient, undesirably slow and result in byproducts that must be occasionally cleaned from the pyrolysis tube and other parts of the deposition equipment of which the pyrolysis tube is a part.

Because of the inefficiencies of the pyrolysis tubes of conventional material processing equipment (e.g., chemical vapor deposition (CVD) equipment, etc.) for depositing parylene, it typically takes several hours (e.g., three hours or longer) to deposit parylene to thicknesses of about 1 micrometer (μm) to about 18 μm or more.

### SUMMARY

This disclosure relates to pyrolysis tubes that are configured to efficiently crack parylene dimers and other materials, as well as to material processing equipment that includes such a pyrolysis tube, and to pyrolysis methods.

In one aspect, a pyrolysis tube according to this disclosure includes a primary conduit, which comprises a primary passage through the pyrolysis tube. The shape, dimensions and area of cross-sections taken transverse to the length of the primary passage may be uniform or substantially uniform (e.g., accounting for manufacturing tolerances, etc.) along the entire length of the pyrolysis tube. The primary passage is effectively subdivided into a plurality of sub-conduits, or channels. Accordingly, such a pyrolysis tube may be referred to as a “multi-channel pyrolysis tube.” In some embodiments, the longitudinal axes of the channels may be oriented parallel to one another, and parallel to the longitudinal axis of the primary passage, which may enable materials to flow directly through the lengths of the channels and, thus, through the primary passage of the pyrolysis tube. In other embodiments, the longitudinal axes of the channels may be configured to provide less direct flow paths. Without limitation, a channel may be curved, or even helical.

All of the channels may extend along the entire length of the primary conduit. Alternatively, one or more—even all—of the channels may extend only partially along the length of the primary conduit. Each channel may have a substantially uniform cross-sectional shape, substantially uniform dimensions and a substantially uniform area along its entire length.

The channels through a pyrolysis tube may be defined by one or more elongated elements that extend through at least a portion of the length of the pyrolysis tube. These elongated elements are referred to herein as “internal elements.” The internal elements may be embodied as one or more tubes that extend at least partially through the length of the primary passage of the pyrolysis tube. As another option, one or more of the internal elements of a pyrolysis tube may comprise a divider that extends across the primary conduit and at least partially along the length of the primary conduit or along the lengths of any other structures that may define channels through the primary conduit of the pyrolysis tube. In various embodiments, an internal element may be formed as an integral part of the pyrolysis tube, an internal element may be secured to one or more other internal elements and/or within (e.g., by welding, brazing, interference fit, etc.) the primary conduit through the pyrolysis tube or an internal element or an assembly of internal elements may comprise an insert that may be placed within and removed from the primary conduit of the pyrolysis tube.

The internal elements that define the channels within a pyrolysis tube according to this disclosure may be formed by a material that will withstand the conditions (e.g., the high temperatures, etc.) of pyrolysis. In some embodiments, the material from which the channel-defining elements of a pyrolysis tube are formed may comprise a thermally conductive material. Elements that are formed from a thermally conductive material may be continuous with, contact or otherwise convey heat from the outer wall of the pyrolysis tube, which defines the primary conduit through the pyrolysis tube, and improve the efficiency with which the heat is radiated throughout the interior of the pyrolysis tube.

A pyrolysis tube may be configured to distribute heat uniformly or substantially uniformly (i.e., within a certain range (e.g., twenty percent, ten percent, five percent, etc.) of the average temperature of the surfaces of the outer wall of the pyrolysis tube, etc.) throughout the interior of the pyrolysis tube.

A pyrolysis tube configured in accordance with teachings of this disclosure may enable pyrolysis to occur efficiently at a lower-than-conventional temperature (e.g., a temperature of less than 680° C., a temperature of 550° C. to 680° C., a temperature of less than 550° C., a temperature of less than 500° C., a temperature of about 400° C. to about 450° C., etc.). Such a configuration may also facilitate the use of smaller, or shorter, pyrolysis tubes. In addition, such a configuration may decrease the time required to effectively pyrolyze a parylene dimer and, thus, decrease the overall duration of time needed to deposit a parylene film of any desired thickness onto a substrate. By enabling the use of lower pyrolysis temperatures and increasing the efficiency with which parylene dimers are pyrolyzed, uniformity or substantially uniformity of the temperature across the primary passage through the pyrolysis tube may also decrease the frequency with which the pyrolysis tube or elements downstream from the pyrolysis tube are cleaned.

In another aspect, a material deposition system or another embodiment of material processing equipment may include a pyrolysis tube according to this disclosure. In some embodiments, the material processing equipment may comprise conventional material processing equipment with a conventionally configured pyrolysis tube. An insert for the pyrolysis tube may be configured to impart the pyrolysis tube with a plurality of channels. The insert may be configured to be introduced into and removed from a primary conduit of the conventionally configured pyrolysis tube. The use of an insert with a conventionally configured pyrolysis tube of conventional material processing equipment may improve the efficiency with which the pyrolysis tube pyrolyzes precursor material, enable the conventional material processing equipment to complete pyrolysis in a reduced duration of time and/or enable the conventional material processing equipment to operate at a reduced pyrolysis temperature.

In embodiments where the insert is configured to be removed from the pyrolysis tube, removability of the insert may enable inserts with a plurality of different configurations to be used with the same pyrolysis tube, as well as cleaning of the insert and/or the pyrolysis tube.

In other embodiments, material processing equipment may include a multi-channel pyrolysis tube with fixed internal elements.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings:

FIG. 1 is a schematic representation of a material deposition system with which a pyrolysis tube according to this disclosure may be used;

FIGS. 2 and 2A are respectively an end view and a perspective view of an embodiment of pyrolysis tube that includes a plurality of channels;

FIGS. 3 and 3A are an end view and a perspective view, respectively, of another embodiment of pyrolysis tube with a plurality of channels;

FIG. 4 is an end view of yet another embodiment of pyrolysis tube that includes a plurality of channels;

FIG. 5 is a perspective view of an end portion of another embodiment of pyrolysis tube with a plurality of channels;

FIG. 5A illustrates an embodiment of multi-channel pyrolysis tube with a flow enhancer on at least one of its ends; and

FIG. 6 is a graph illustrating an increase in the thicknesses of a polymer film that may achieved when a material deposition system that employs a pyrolysis tube according to this disclosure (as opposed to a conventional pyrolysis tube) is used to deposit the polymer film.

### DETAILED DESCRIPTION

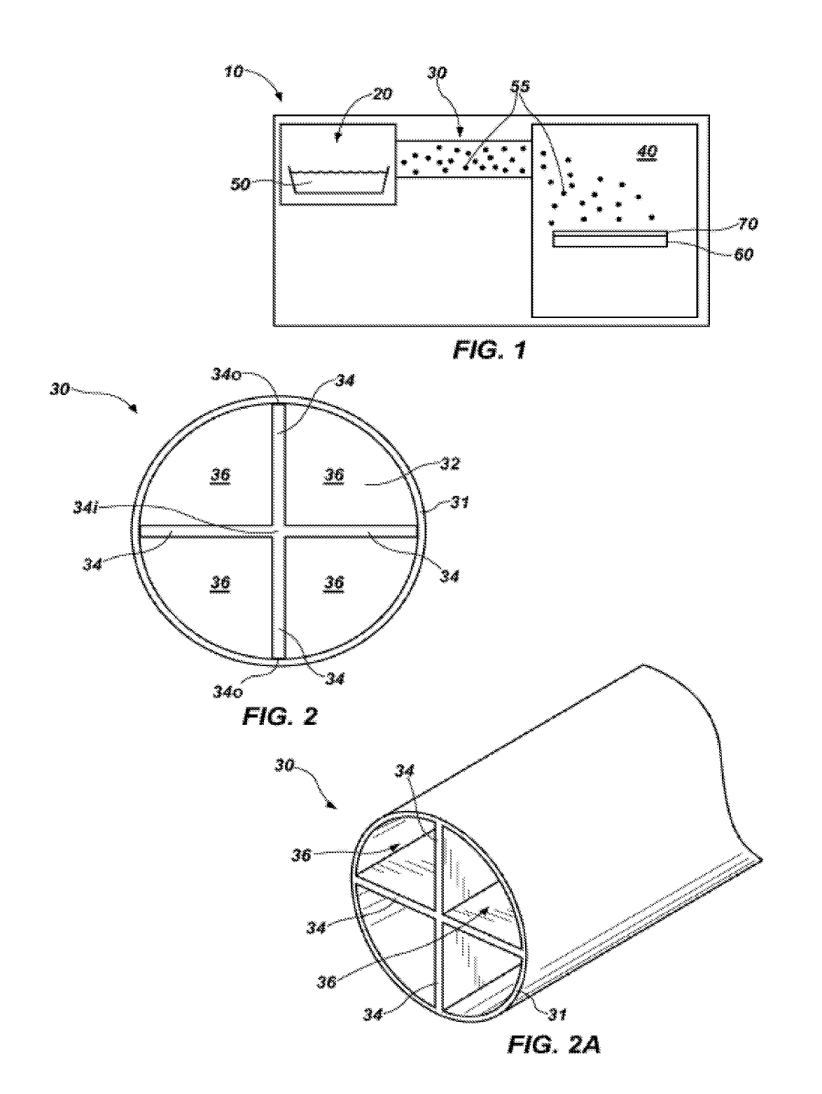

With reference to FIG. 1, a schematic representation of a material deposition system 10 is illustrated. The depicted embodiment of material deposition system 10 includes a pyrolysis tube 30 and a deposition chamber 40 downstream from the pyrolysis tube 30. Additionally, the material deposition system 10 may include a volatilization element 20, such as a vaporization chamber, upstream from the pyrolysis tube 30.

In use, the material deposition system 10 may be configured to receive a precursor material 50, convert the precursor material 50 to reactive species 55 and provide an environment in which molecules of the reactive species 55 may react with one another to form a polymer film 70 on one or more substrates 60. In the specific embodiment depicted by FIG. 1, a precursor material 50, such as a substituted or unsubstituted parylene dimer (e.g., [2.2]paracyclophane, etc.), may be placed in the volatilization element 20 of the material deposition system 10. The volatilization element 20 may be configured to introduce the precursor material 50 into the pyrolysis tube 30. As a non-limiting example, the volatilization element 20 may be configured to be heated to a temperature that will vaporize, sublimate or otherwise volatilize the precursor material 50. As volatilized precursor material enters the pyrolysis tube 30, the volatilized precursor material 50 may be heated to a temperature that will “crack” the precursor material 50 into reactive species 55 (e.g., substituted or unsubstituted p-xylylene, etc.). The reactive species 55 may then be communicated (e.g., drawn under a vacuum, etc.) into the deposition chamber 40, which may provide conditions that enable molecules of the reactive species 55 to react with one another and to form a polymer film 70 on exposed surfaces of one or more substrates 60 within the deposition chamber 40.

In a variety of embodiments, including that depicted by FIG. 2, the pyrolysis tube 30 of a material deposition system 10 may comprise an elongated element with a primary conduit 32 extending through its length. In some embodiments, the primary conduit 32 of the pyrolysis tube 30 may have a cylindrical configuration. A plurality of internal elements 34, which may also be referred to as “inserts,” may comprise elongated elements that extend through the length of the primary conduit 32, dividing the primary conduit 32 into a plurality of sub-conduits, or channels 36. The internal elements 34 may be configured to define at least some channels 36 that are linear and, therefore, provide direct lines of sight through the pyrolysis tube 30.

FIGS. 2 through 5 depict some specific embodiments of pyrolysis tubes that may be used in a material deposition system, such as a material deposition system 10 that includes the element depicted by FIG. 1.

The embodiment of pyrolysis tube 30 shown in FIGS. 2 and 2A includes an outer wall 31. The outer wall 31 defines a primary conduit 32 through the length of the pyrolysis tube 30. The pyrolysis tube 30 also includes a plurality of radially oriented internal elements 34. Each internal element 34 includes an outer edge 34o adjacent to (or, optionally, secured to or continuous with) an interior surface of the outer wall 31 of the pyrolysis tube 30. Internal edges 34i of the internal elements 34 meet at a somewhat central location within the primary conduit 32. In the depicted embodiments, the internal edges 34i of the internal elements 34 meet along a central axis 32a of the primary conduit 32 and of the pyrolysis tube 30. The specific embodiment of pyrolysis tube 30 illustrated by FIGS. 2 and 2A includes four internal elements 34, which divide the primary conduit 32 into four sub-conduits, or channels 36. Of course, pyrolysis tubes with other numbers of internal elements and channels (e.g., 5, 6, 7, 8, 10, etc.) are also within the scope of this disclosure.

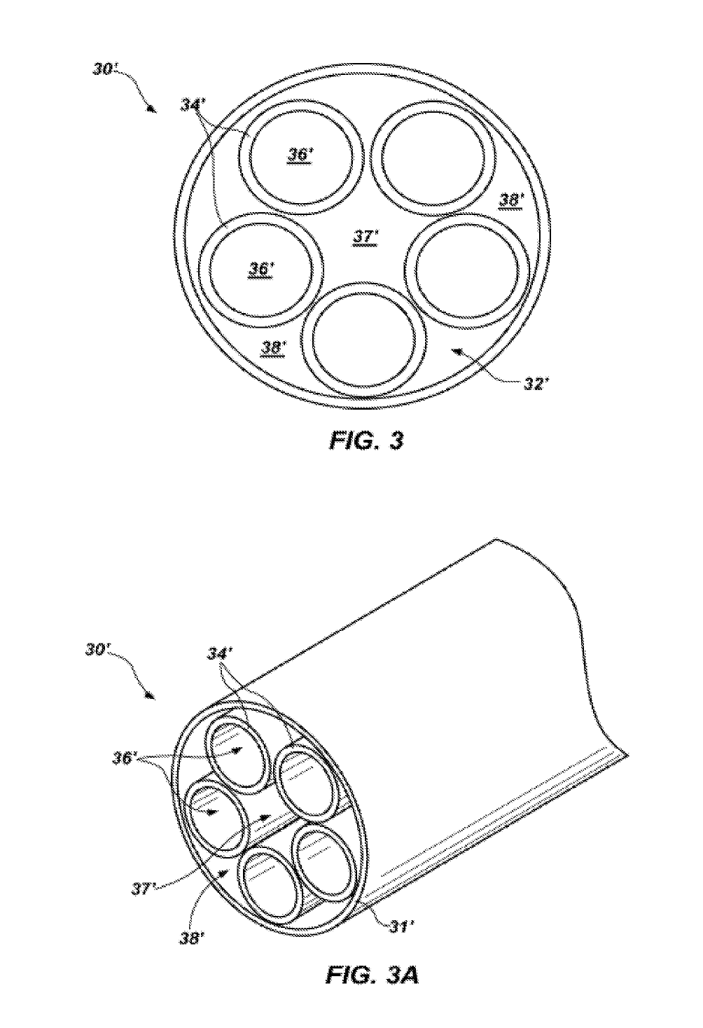

FIGS. 3 and 3A illustrate an embodiment of pyrolysis tube 30′ that includes a plurality of internal tubes 34′ within its primary conduit 32′. The internal tubes 34′ may be arranged around an interior periphery of the pyrolysis tube 30′, as defined by an outer wall 31′ of the pyrolysis tube 30′. In the embodiment depicted by FIGS. 3 and 3A, five internal tubes 34′ are arranged in a somewhat pentagonal arrangement around the interior periphery of the pyrolysis tube 30′. Each internal tube 34′ includes a conduit that defines a sub-conduit, or channel 36′ through the pyrolysis tube 30′. In the depicted embodiment, the internal tubes 34′ and the channels 36′ are cylindrical in shape. In addition to the channel 36′ defined through each internal tube 34′, a central channel 37′ may be defined between a group of internal tubes 34′ and a peripheral channel 38′ may be defined between each adjacent pair of internal tubes 34′ and an adjacent portion of the interior of an outer wall 31′ of the pyrolysis tube 30′.

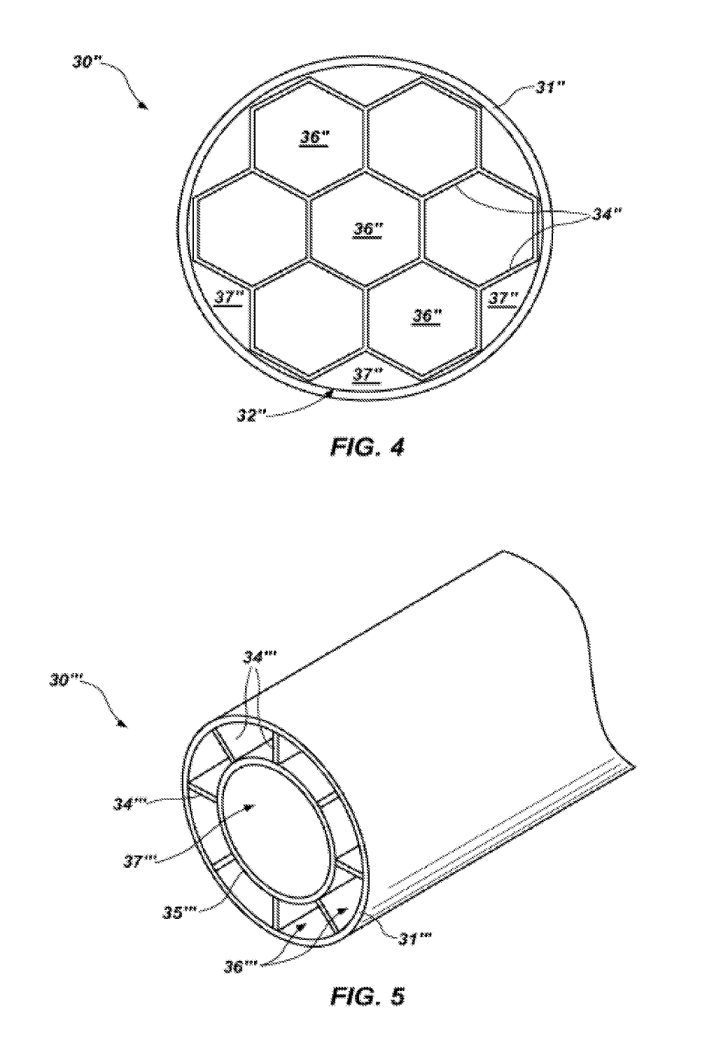

In FIG. 4, an embodiment of pyrolysis tube 30″ is depicted that includes an outer wall 31″, a primary conduit 32″ defined by an interior surface of the outer wall 31″ and a plurality of internal elements 34″ arranged within the primary conduit 32″. The internal elements 34″ comprise flat, elongated elements that are arranged within the primary conduit 32″ in a manner that defines a plurality of channels 36″ with polygonal prismatic shapes through at least a portion of the length of the primary conduit 32″. In some embodiments, the channels 36″ may have the same shapes and configurations (e.g., hexagonal prisms, as depicted; rectangular prisms; triangular prisms; etc.). In addition to channels 36″, smaller peripheral channels 37″ may be defined between an interior surface of the outer wall 31″ of the pyrolysis tube 30″ and one or more adjacent internal elements 34″.

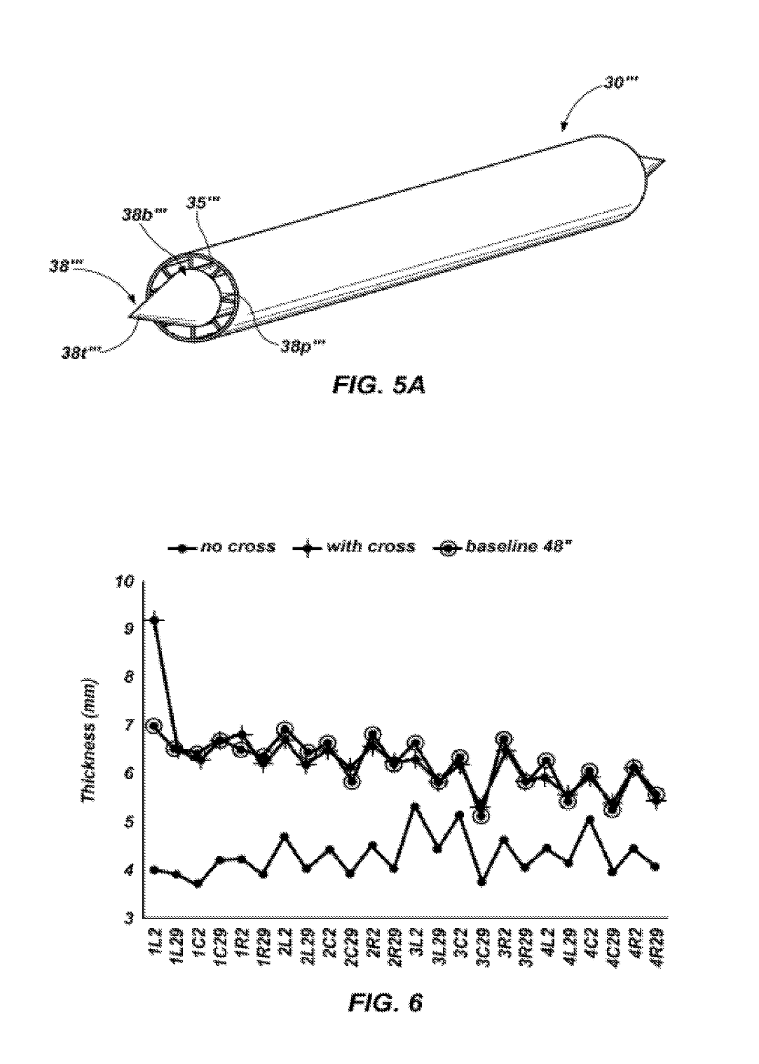

Another embodiment of pyrolysis tube 30″′, which is depicted by FIG. 5, includes an outer wall 31″′, a central interior element 35″′ positioned coaxially with respect to the outer wall 31″′ and a plurality of radially oriented internal elements 34″′. Optionally, the central interior element 35″′ may comprise a tube. In some embodiments, the central interior element 35″′ may be cylindrical in shape. The internal elements 34″′ may be spaced apart from one another and extend between an outer surface of the central interior element 35″′ and an inner surface of the outer wall 31″′. A channel 36″′ may be defined between each adjacent pair of internal elements 34″′ and the sections of the interior surface of the outer wall 31″′ and the exterior surface of the central interior element 35″′ extending between that adjacent pair of internal elements 34″′. In embodiments where the central interior element 35″′ comprises a tubular element, it may define a central channel 37″′ through the pyrolysis tube 30″′.

In some embodiments, such as that depicted by FIG. 5A, a flow enhancer 38″′ may be positioned adjacent to one end or each end of the central interior element 35″′. Each flow enhancer 38″′ may taper from a relatively large dimension at its base 38b″′ to a smaller dimension (e.g., a point, etc.) at its tip 38t″′. A periphery 38p″′ of a base 38b″′ of the flow enhancer 38″′ may be configured similar to or the same as a cross-sectional shape of the central interior element 35″′, taken along a length of the central interior element 35″′ (e.g., a circular cross-sectional shape, a polygonal cross-sectional shape, etc.). One or both of the taper of the flow enhancer 38″′ and the shape of its base 38b″′ may reduce the friction with which gases and/or materials flow into and/or out of the channels 36″′ of the pyrolysis tube 30″′. A configuration such as that depicted by FIG. 5A may effectively, but not actually, reduce the size of a pyrolysis tube 30″′, enabling the use of a relatively larger (e.g., 3 inch (about 7.8 cm), etc.) diameter pyrolysis tube to simulate a relatively smaller (e.g., 1.5 inch (about 3.9 cm), etc.) diameter pyrolysis tube.

The internal elements of a pyrolysis tube that incorporates teachings of this disclosure, as well as the shapes of the channels that are defined by the internal elements, may be configured to increase surface area within the pyrolysis tube and, thus, the likelihood that molecules of precursor material will collide with a surface or another molecule of precursor material within the pyrolysis tube. While the internal elements and the channels of a pyrolysis tube according to this disclosure may be configured to increase the surface area within the pyrolysis tube, they may also be configured not to interrupt or impede the flow of a precursor material and/or reactive species formed therefrom through the pyrolysis tube. As depicted by FIGS. 2 through 5A, each channel 36, 36′, 36″, 36″′ of a pyrolysis tube 30, 30′, 30″, 30″′ may extend linearly through the length of the pyrolysis tube 30 and, thus provide a direct line of sight through the pyrolysis tube 30. By increasing the surface area of the pyrolysis tube while providing a direct line of sight to the source of radiation (e.g., the inner diameter of the pyrolysis tube, etc.), the frequency with which precursor molecules will directly contact a heated surface of the pyrolysis tube will increase, in turn increasing the efficiency with which the precursor material will be cracked and, further, increasing the rate of pyrolysis. Alternatively, one or more channels of a pyrolysis tube with two or more channels may follow a helical path through at least a portion of the length of the pyrolysis tube. In a more specific embodiment, a pyrolysis tube may include a plurality of helical channels, each of which may rotate, or twist 90°, or a quarter of a turn, along the length of the pyrolysis tube. Of course, helically configured channels with less of a twist or with more of a twist are also within the scope of this disclosure. In addition, pyrolysis tubes that include one or more linear channels and one or more helical channels are within the scope of this disclosure.

The internal elements of a pyrolysis tube according to this disclosure (e.g., internal elements 34 (FIGS. 2 and 2A), internal tubes 34′ (FIGS. 3 and 3A), internal elements 34″ (FIG. 4); internal elements 34″′ and central interior element 35″′ (FIG. 5), etc.) may comprise an integral part of the pyrolysis tube, or they may comprise an insert that is configured to be introduced into and readily removed from a primary channel of a conventionally configured pyrolysis tube (e.g., for ease in cleaning, to enable enhancement of a pyrolysis tube of an existing material deposition system, etc.).

Any suitable materials that will withstand the conditions of pyrolysis (e.g., temperatures of 400° C. or greater, temperatures of 500° C. or greater, temperatures of 600° C. or greater, temperatures of 700° C. or greater, temperatures of up to 800° C., etc.) may be used to form the internal elements of a pyrolysis tube or an insert according to this disclosure. A few non-limiting examples of suitable materials include steel, stainless steel, aluminum, an austenitic nickel-chromium-based super alloy, such as those available from Special Metals Corporation of New Hartford, N.Y., under the trademark INCONEL®, cobalt-chrome, titanium, silver and gold.

Experimentation revealed several indicators of the extent to which a pyrolysis tube 30 with a plurality of channels extending along at least a portion of its length improves the efficiency with which a precursor material 50 is cracked into reactive species 55. In the experiment, the performance of a pyrolysis tube 30 having the configuration shown in FIGS. 2 and 2A, with a length of 30 inches (about 76 cm) and a diameter of 1.5 inches (about 3.8 cm) (the “multi-channel pyrolysis tube”) was compared with conventional cylindrical pyrolysis tubes having diameters of 1.5 inches (about 3.8 cm) and lengths of 30 inches (about 76 cm) and 48 inches (about 122 cm).

Each pyrolysis tube was used to deposit a film of Parylene C onto a substrate under so-called “under-cracking” conditions, in which the precursor material (500 g of Parylene C dimer was used with each test of each pyrolysis tube) (i.e., Parylene C dimer) would be expected to condense at the entry point to the deposition chamber 40 (FIG. 1) of the material deposition system 10 and undesirably thin polymer films 70 (FIG. 1) would be expected to form on the substrates 60 (FIG. 1). To achieve these conditions, the volatilization element 20 was heated to a temperature of 180° C. and each pyrolysis tube was heated to a relatively low temperature (575° C. for the multi-channel pyrolysis tube and the 30 inch conventional cylindrical pyrolysis tube and 600° C. for the 48 inch conventional cylindrical pyrolysis tube).

A variety of results were analyzed. As shown by the graph of FIG. 6, the polymer films that were deposited when the multi-channel pyrolysis tube was used had about the same thicknesses as the polymer films that were deposited when the longer, 48 inch pyrolysis tube was used at a higher temperature. In contrast, the polymer films that were deposited when the 30 inch conventional cylindrical pyrolysis tube was used were only about two-thirds as thick. These results indicate that a multi-channel pyrolysis tube is about 50% more efficient than a conventional pyrolysis tube of the same outer configuration and length.

In addition, observations of the entry point to the deposition chamber showed that little or no precursor material condensation was present when the multi-channel pyrolysis tube was used, while a significant amount of precursor material condensed at the entry point to the deposition chamber when the 30 inch conventional cylindrical pyrolysis tube was used. These results indicate that even when pyrolysis was conducted at a relatively low temperature, there was little or no under-cracking of the precursor material when the multi-channel pyrolysis tube was used. Thus, it appears that the multi-channel pyrolysis tube cracked molecules of the precursor material with greater efficiency than the conventionally configured pyrolysis tube. The improved cracking efficiency may reduce the frequency with which precursor material accumulates within the pyrolysis tube, which may reduce the frequency with which the pyrolysis tube should be cleaned, relative to the frequency with which conventionally configured pyrolysis tubes are cleaned.

Observations of the entry point to the deposition chamber also indicated that there may have been little or no over-cracking of the precursor material. Under the specific test parameters identified above, experimental results have shown that significant over-cracking, which includes the removal of chlorine (Cl) atoms from the Parylene C dimer, may result in a film that is green in color. No green color was present at the entry point to the deposition chamber.

These results support the belief that heat from radiation cannot completely crack molecules of precursor material by itself; an increased number of collisions between the molecules of precursor material increase the rate at which cracking occurs and, thus, the efficiency with which molecules of the precursor material are cracked. By separating the primary conduit through a pyrolysis tube into a plurality of channels, the rate at which collisions occur between molecules of precursor material is increased, which may lead to an increased rate of cracking, and to the increased efficiencies that were observed from the results of the above-described experimentation.

The results of the above-described experimentation also indicate that when a pyrolysis tube with a plurality of channels is used in a material deposition process, efficient and effective pyrolysis may occur at a relatively low temperature (e.g., 600° C., 575° C., 550° C., 500° C., 450° C., 425° C., etc., or less). They also suggest that, when higher (e.g., conventional, etc.) pyrolysis temperatures are used, the process of cracking molecules of a precursor material into reactive species may occur at a higher rate, which may also result in faster polymerization and deposition rates, and the deposition of a polymer film of a given thickness in a reduced amount of time.

As another option, by imparting a pyrolysis tube with a multi-channel configuration, its length may be shortened or effectively shortened (e.g., less of its length may be heated, etc.), which may reduce the size and cost of material deposition systems and/or the cost of operating material deposition systems (e.g., the energy required to heat the pyrolysis tube is decreased, etc.).

Although the foregoing disclosure provides many specifics, these should not be construed as limiting the scope of any of the appended claims, but merely as providing information pertinent to some specific embodiments that may fall within the scopes of the claims. Other embodiments may be devised which lie within the scopes of the claims. Features from different embodiments may be employed in any combination. All additions, deletions and modifications, as disclosed herein, that fall within the scopes of the claims are to be embraced by the claims.

1-30. (canceled)

31. A pyrolysis tube for a material deposition system, comprising:
an outer body through which a primary conduit is defined, the outer body comprising a material that can be heated to at least a pyrolysis temperature sufficient to pyrolyze a material to be deposited onto a substrate;
at least one internal element within the primary conduit, dividing the primary conduit into channels, all of the channels in the primary conduit providing a path through an entirety of a length of the primary conduit, the least one internal element comprising a material configured to be heated to at least the pyrolysis temperature, the at least one internal element configured to be received by and removed from the primary conduit of the outer body; and
a flow modifier positioned adjacent to an end of the at least one internal element, the flow modifier configured to effectively reduce an overall size of the pyrolysis tube.

32. The pyrolysis tube of claim 31, wherein the at least one internal element defines a central channel extending along the length of the primary conduit and positioned centrally within the primary conduit.

33. The pyrolysis tube of claim 32, wherein the central conduit and the primary conduit are coaxial with one another.

34. The pyrolysis tube of claim 32, comprising a plurality of internal elements, each internal element of the plurality extending longitudinally along a length of the primary conduit and radially from an inner surface of the outer body to an outer surface of the central channel.

35. The pyrolysis tube of claim 34, wherein the plurality of internal elements are arranged to define a plurality of congruent channels through the length of the primary conduit.

36. The pyrolysis tube of claim 35, the plurality of congruent channels having substantially the same cross-sectional shape and dimensions.

37. The pyrolysis tube of claim 32, wherein the flow modifier is positioned adjacent to an end of the central channel.

38. The pyrolysis tube of claim 31, wherein the at least one internal element intersects a longitudinal axis through a center of the length of the primary conduit.

39. An insert for a pyrolysis tube of a material deposition system, comprising:
at least one internal element configured to be inserted into a primary conduit of a pyrolysis tube of a material deposition system, to extend along at least a portion of a length of the primary conduit and to divide the primary conduit into a plurality of elongate channels; and
a flow modifier positioned adjacent to an end of the at least one internal element, the flow modifier configured to effectively reduce an overall size of the pyrolysis tube.

40. The insert of claim 39, wherein the at least one internal element comprises an inner tube configured to be oriented coaxially with the pyrolysis tube.

41. The insert of claim 40, wherein the at least one internal element comprises a plurality of internal elements configured to hold the inner tube in place within the primary conduit of the pyrolysis tube.

42. The insert of claim 41, wherein the plurality of internal elements extend radially outward from an exterior surface of the inner tube to an inner surface of the pyrolysis tube.

43. The insert of claim 40, wherein the flow modifier is positioned adjacent to an end of the inner tube.

44. The insert of claim 39, wherein the at least one internal element comprises a plurality of internal elements arranged to define a plurality of columns having polygonal prismatic configurations.

45. A material deposition system, comprising:
a pyrolysis tube;
an insert configured to be placed within and removed from the pyrolysis tube to define channels extending along an entirety of a length of the pyrolysis tube;
a deposition chamber in communication with the pyrolysis tube; and
a flow modifier positioned adjacent to an end of the insert, the flow modifier configured to effectively reduce an overall size of the pyrolysis tube.

46. The material deposition system of claim 45, wherein the insert comprises:
an inner tube configured to be oriented coaxially with the pyrolysis tube.

47. The material deposition system of claim 46, wherein the flow modifier is positioned adjacent to an end of the inner tube.

48. The material deposition system of claim 46, wherein the pyrolysis tube comprises an existing pyrolysis tube of a material deposition system.

49. The material deposition system of claim 48, wherein the insert is configured to enable a reduction in a temperature to which the material deposition system is configured to heat the pyrolysis tube.

50. The material deposition system of claim 48, wherein the insert is configured to enable a reduction in a frequency with which the pyrolysis tube is cleaned.
